(19) United States
(12) Patent Application Publication
(10) Pub. No.: US 2026/0076708 A1
(43) Pub. Date: Mar. 19, 2026

(54) CUTTING DEVICE AND RELATED SYSTEMS AND METHODS

(71) Applicant: GWBN, LLC, Arnold, MD (US)

(72) Inventors: Glenn WHITMAN, Baltimore, MD (US); William NILAND, Arnold, MD (US); Peter WILSON, Killingworth, CT (US)

(21) Appl. No.: 19/401,275

(22) Filed: Nov. 25, 2025

Related U.S. Application Data
(63) Continuation of application No. PCT/US2024/030787, filed on May 23, 2024.
(60) Provisional application No. 63/504,460, filed on May 26, 2023.

(51) Int. Cl. A61B 17/3207; A61B 17/00; A61M 29/00

(52) U.S. Cl. CPC A61B 17/3207; A61M 29/00; A61B 2017/00477

(57) ABSTRACT

Systems, apparatus, and methods are described for forming an incision in tissue for receiving a surgical instrument. A cutting device for forming the incision can be reversibly coupleable to an instrument, e.g., a dilator. The instrument may define a lumen configured to receive a wire that extends or is otherwise extendable through a puncture site. The cutting device can include a cutting element configured to be exposed by a guard to form the incision such that the incision extends form the puncture site and is sized to receive the instrument.

### CROSS-REFERENCE TO RELATED APPLICATIONS

This application is a continuation of International Patent Application No. PCT/US2024/030787, filed May 23, 2024, titled “CUTTING DEVICES AND RELATED SYSTEMS AND METHODS,” which claims priority to and the benefit of U.S. Provisional Ser. No. 63/504,460 , filed May 26, 2023, titled “CUTTING DEVICES AND RELATED SYSTEMS AND METHODS,” the disclosures of which are incorporated herein by reference.

### TECHNICAL FIELD

The present disclosure relates generally to systems, apparatus, and methods for deploying a medical instrument into a body lumen of a subject during a medical procedure, and in particular, to a cutting device capable of forming an incision for receiving a medical instrument, such as a catheter, into a body lumen of a subject.

### BACKGROUND

Intravascular medical devices such as catheters are deployed in many medical procedures. Use of intravascular catheters, however, can lead to bloodstream infections, which can be costly to treat and/or result in death or other health complications. For example, an infection can result from skin organisms that migrate from an insertion site of a catheter onto and along an external surface of the catheter. This migration of skin organisms along the catheter which dwell within a central vessel, artery, or vein, can lead to a blood stream infection. In many hospitals in the U.S. including high performing intensive care units, this type of event occurs approximately 1-3 times every 1000 central line days, and sometimes far more. Infections can also be tied to use of other types of catheters placed for other reasons to provide medical care, including catheters such as dialysis catheters, cannulation catheters for extracorporeal membrane oxygenation (ECMO), and chest tubes placed within the pleural cavity.

A catheter or other intravascular medical device can be delivered into blood vessels, organs, body cavities, and other anatomic sites (“target site(s)” or “target anatomical site(s)”) using a variety of techniques. One commonly used technique to gain access to a target site (e.g., a blood vessel) is the Seldinger technique. The Seldinger technique involves penetrating through skin tissue overlying a blood vessel of a subject with a sharp hollow object, typically a hollow needle. A wire (e.g., a guidewire) can then be advanced via a lumen of the needle into the blood vessel, and the needle can be withdrawn over the guidewire and removed.

Following placement of the wire but prior to insertion of the catheter or other intravascular device, an incision (e.g., “skin-nick,” in cases where the diameter of the catheter is small) in the skin tissue is formed through the skin at or adjacent to the opening formed by the needle (i.e., the entry site for the wire). If formed properly, the incision will start at the opening or puncture site of the needle, and have a length approximately equal to half of the perimeter of the catheter to be subsequently inserted. After the incision is formed, the catheter or other medical instrument can then be passed over the wire, through the incision, and into the blood vessel or body cavity.

While techniques such as the Seldinger technique can be used to deploy a catheter or other medical instrument into a target site, such as a blood vessel, they can be difficult and/or time-consuming to perform properly. For example, complications can occur with the creation of the incision, e.g., where the incision does not initiate at the opening of the puncture site resulting in a skin-bridge, the incision is too large, or the incision is too long. When these complications arise, it may be impossible (or additional measures may be required) to insert the dilator or catheter (or other medical instrument(s)) over the Seldinger wire into the target vessel, such as in the case of a skin bridge). Alternatively, if an incision is too large, bleeding may occur around the catheter, which can be substantial, or the incision itself, being open, can provide a site for colonization, either of which increases the risk of bacterial colonization at the catheter entry site, increasing the risk of a blood stream infection. Further issues can also affect incision. Poor visibility, blood coming back around the guidewire at the skin insertion site, or movement of the patient during cannulation (e.g. cardiac arrest with ongoing CPR) can make accurate incisions very difficult. Accordingly, it is desirable to have systems and methods that reduces the difficulty and/or skill required to gain access to a target site, thereby reducing complications associated with use of any invasive catheter.

### SUMMARY

According to an aspect of the present disclosure, an apparatus includes a cutting device. The cutting device includes a body defining a longitudinal axis and a plurality of couplers configured to reversibly couple the cutting device to a dilator that is disposable about a guidewire that has been inserted into a puncture site formed in tissue, a cutting element coupled to a distal end of the body and extending distally therefrom, the cutting element including a cutting edge that is angled with respect to the longitudinal axis by an angle of less than about 45 degrees, a distal end of the cutting element extending distally beyond a distal end of dilator and at an angle toward the guidewire when the cutting device is coupled to the dilator via the plurality of couplers. The cutting element further includes a guard slidably coupled to the body, the guard configured to move between an extended position in which the guard covers the cutting element and a retracted position in which the cutting edge of the cutting element is exposed and configured to form an incision in the tissue that is connected to and extends from the puncture site.

According to an aspect of the present disclosure, an apparatus includes a cutting device. The cutting device includes a body defining a longitudinal axis and a plurality of couplers configured to reversibly couple the cutting device to a dilator that is disposable about a guidewire that has been inserted into a puncture site formed in tissue. The cutting device further includes a cutting element coupled to a distal end of the body and extending distally therefrom, the cutting element including first, second, and third edges, the first and second edges being cutting edges that face away from the dilator when the cutting device is coupled to the dilator via the plurality of couplers, the third edge being on an opposite side of the cutting element than the first and second edges, each of the first, second, and third edges being angled with respect to the longitudinal axis. The cutting device further includes a guard slidably coupled to the body, the guard configured to move between an extended position in which the guard covers the cutting element and a retracted position in which at least a portion of the first and second edges is exposed and configured to form an incision in the tissue that is connected to and extends from the puncture site.

According to an aspect of the present disclosure, an apparatus includes a cutting device. The cutting device includes a body defining a longitudinal axis and a plurality of couplers configured to reversibly couple the cutting device to a dilator that is disposable about a guidewire that has been inserted into a puncture site formed in tissue. The cutting device further includes a cutting element coupled to a distal end of the body and extending distally therefrom, the cutting element including an outer cutting edge and an inner edge that are angled with respect to the longitudinal axis. The cutting device further includes a guard slidably coupled to the body, the guard configured to move between an extended position in which the guard covers the cutting element and a retracted position in which the cutting edge of the cutting element is exposed and configured to form an incision in the tissue that is connected to and extends from the puncture site, the guard including an indicator that can be aligned with a distal end of the dilator when the guard is in the extended position such that, when the cutting device is coupled to the dilator via the plurality of couplers, a distal end of the cutting element extends distally beyond the distal end of dilator by a distance of less than about 3 mm. The cutting device further includes an actuator coupled to the guard, the actuator configured to be depressed to allow the guard to move from the extended position to the retracted position and to be released after the guard is in the retracted position to lock the guard to the body via a mechanical engagement between the guard and the body.

According to an aspect of the present disclosure, a method includes advancing a dilator, over a wire positioned through a puncture site formed in a tissue, toward the puncture site, the dilator having reversibly coupled thereto a cutting device including a cutting element configured to cut the tissue and a guard configured to cover the cutting element. The method includes moving the guard from an extended position in which the guard is covering the cutting element to a retracted position in which a portion of the cutting element including a cutting edge is exposed, the cutting edge being at an angle relative to a longitudinal axis of the cutting device that is less than about 45 degrees. The method includes inserting, after moving the guard to the retracted position, the distal end of the dilator and the cutting element into the tissue such that the cutting element forms an incision extending from the puncture site and sized to receive a surgical instrument.

It should be appreciated that all combinations of the foregoing concepts and additional concepts discussed in greater detail below (provided such concepts are not mutually inconsistent or exclusive) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Other systems, processes, and features will become apparent to those skilled in the art upon examination of the following drawings and detailed description. It is intended that all such additional systems, processes, and features be included within this description, be within the scope of the present disclosure, and be protected by the accompanying claims.

### BRIEF DESCRIPTION OF THE DRAWINGS

The drawings are not necessarily to scale. The drawings are merely schematic representations, not intended to portray specific parameters of the invention. The drawings are intended to depict only typical embodiments of disclosed systems, apparatus, and methods. In the drawings, like reference characters refer to like elements (e.g., functionally similar and/or structurally similar elements).

FIG. 1 is a schematic diagram depicting a cutting device, according to an embodiment.

FIG. 2 is a schematic diagram depicting a cutting device and a medical device with which the cutting device can be used, according to an embodiment.

FIG. 3 is a flow diagram illustrating a method of performing a medical procedure on a subject using a cutting device, according to an embodiment.

FIG. 4 is a schematic diagram depicting a kit including a cutting device, according to an embodiment.

FIGS. 5A and 5B schematically depict a puncture site and incision along a skin surface.

FIG. 6A depicts an enlarged view of a distal end of a cutting device, according to an embodiment. FIG. 6B depicts a front view of the cutting device of FIG. 6A.

FIG. 7 depicts a perspective view of a cutting device, according to embodiments.

FIG. 8 depicts an exploded view of the cutting device shown in FIG. 7, according to embodiments.

FIGS. 9A and 9B depict side views of the cutting device shown in FIG. 7, in two different configurations, according to embodiments.

FIG. 10A depicts a side view of the cutting device shown in FIG. 7, with an outer guard removed to show interior components of the cutting device, according to embodiments.

FIG. 10B depicts a view of a distal end of the cutting device shown in FIG. 24, according to embodiments.

FIG. 11 depicts a perspective view of the cutting device shown in FIG. 24, coupled to a dilator and guidewire, according to embodiments.

FIG. 12 depicts a side view of a cutting device, according to embodiments.

FIGS. 13A-13C depict side views of a retraction of a guard of the cutting device of FIG. 12, according to embodiments.

FIG. 13D depicts a close up side view of a distal end of the cutting device of FIG. 12, according to embodiments.

FIG. 14 depicts a bottom view of the cutting device of FIG. 12, according to embodiments.

FIG. 15 depicts a close-up side view of the cutting device of FIG. 12 with a guard with an indicator line, according to embodiments.

FIG. 16 depicts a cutting element with dual cutting edges, according to embodiments.

### DETAILED DESCRIPTION

Embodiments of the present disclosure are directed to systems, apparatus, and methods for providing access to a target site (e.g., a blood vessel) such that a medical instrument, e.g., a catheter, can be deployed in the target site. Specifically, systems, apparatus, and methods described herein can be used to form an incision in tissue of a subject during a vascular access procedure and/or other procedures involving placement of a medical device into a body cavity.

Vascular access procedures, can employ, for example, the Seldinger technique to provide access to a target body lumen (e.g., vessel, pleural space) by deploying a medical instrument (e.g., a catheter) into the target body lumen. For example, when the Seldinger technique is used, a target body lumen (e.g., vessel, pleural space) may be accessed percutaneously by puncturing skin of the subject with a hollow needle, inserting a guidewire through the hollow needle so as to position the distal end of the guidewire in the target body lumen. The needle can be withdrawn, leaving the guidewire in place. To accommodate a placement of a catheter or other medical instrument, the diameter of the skin puncture site traversed by the wire requires enlargement to accommodate the catheter to be placed. To accomplish this, the operator can create a skin incision extending from the puncture site and having a length equal to half of the perimeter of the catheter or other medical instrument. Once that incision is formed, a dilator (or one or more increasingly larger dilators) is passed over the Seldinger wire, creating a track (e.g., opening) suitable to accommodate the final catheter or medical instrument to be inserted into the target body lumen.

The incision can be formed using a cutting device, such as, for example, a blade or scalpel. The physician can hold the guidewire using one hand, and the cutting device using his or her other hand, and then via visual guidance, create the incision, which can receive the dilator, catheter, and/or other medical instrument. If properly performed (e.g., if the incision is properly created or formed), the incision extends from the puncture site and has a length equal to half of the perimeter of the catheter or other instrument to be inserted through the incision. Complications, however, can arise when the incision is not properly formed. For example, when the incision is too long, it allows leakage of bodily fluids (e.g. blood, serum, etc.) around the catheter when it is positioned, allowing blood loss and compromising the ability of the skin to function as a barrier to infection. For example, an incision too long compromises the ability of the skin to form a biological seal around the catheter, which would normally prevent migration of bacteria or other disease-causing agents into the target body lumen. Furthermore, an oversized incision allows blood to leak out around the catheter skin entry site, which can be substantial, particularly in situations where a subject is receiving an anticoagulant. In these cases, the incision may require stitches to better approximate the skin against the catheter so as to prevent bleeding.

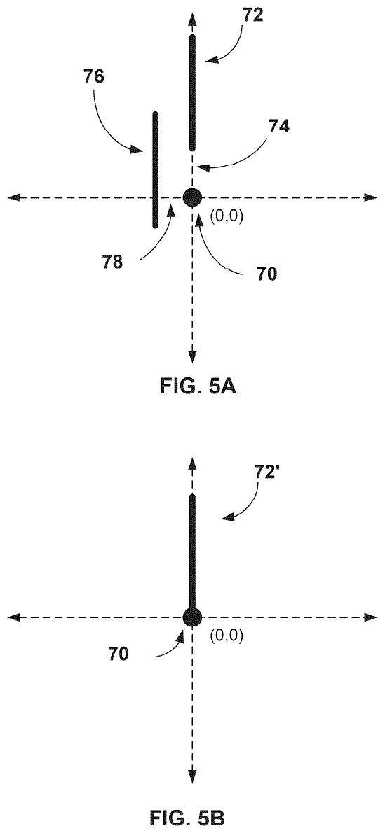

As another example, if the incision does not involve the entry site made by the needle (i.e., in which the Seldinger wire sits), but leaves a skin bridge between the incision and the initial entry site created by the needle, the ensuing dilator and/or other medical instrument can be prevented from being passed along the wire and through the entry site. FIG. 5A schematically illustrates cases where a skin bridge can form. As depicted, skin bridges 574, 578 can form when an incision 572, 576 does not overlap with an opening of the puncture site 570. For example, a skin bridge 574 can form when an incision 572 does not initiate at the puncture site 570, even if that incision is aligned with the puncture site 570. And a skin bridge 578 can form when an incision 576 is offset from the puncture site 570 (e.g., not aligned with the puncture site 570). These skin bridges 574, 578 can prevent a dilator or other instrument from being inserted into the skin. In practice, a physician may move a guidewire after forming the incision 572, 576 to determine whether there is a skin bridge 574, 578, e.g., by visibly inspecting whether the guidewire appears to be moving along a length of the incision. But with skin having some elasticity and with poor visibility surrounding the puncture site (e.g., caused by poor lighting, patient body habitus, or blood pooling around the puncture site and incision), it may not be possible to determine whether a skin bridge has formed even if it appears the guidewire is moving along the incision. If a physician fails to identify that there is a skin bridge 574, 578 and pushes a dilator or other instrument down along the guidewire (e.g., forces a dilator or other instrument down on the guidewire), the physician can bend or damage the guidewire. Then, even if the physician were to detect the skin bridge 574, 578 and cut it away, e.g., with a second incision, the bent guidewire can prevent the dilator or other instrument from being inserted over the guidewire and traversing the skin incision into the skin. Therefore, when complications arise with a skin bridge, and the guidewire is bent, it may be desirable for a physician to remove the guidewire and restart the procedure. Alternatively, the second incision combined with the first incision 572, 576 so as to connect to the puncture site can lead to an incision size that is not sized to that of the dilator or other instrument, but, rather, is too long, which as detailed above, can lead to bleeding and, with time, the subepidermal migration of bacteria along the catheter resulting in a blood stream infection. Systems, devices, and methods described herein are designed to prevent the formation of a skin bridge, i.e., by ensuring that an incision extends from a puncture site, as depicted in FIG. 5B with incision 572′extending from puncture site 570. Further details of such systems, devices, and methods are provided below.

Similarly, if the incisional length is too short, the dilator and/or other medical instrument can be prevented from passing deeper than the level of the skin. If an operator does not recognize a too short incisional length and pushes the dilator sufficiently, the guidewire may bent, thus preventing its use as the dilator may not be able to pass over the bent wire, even after the skin incision has been lengthened.

In the situations described above, if the problem is initially unrecognized, forcing the dilator can bend the wire or damage the dilator and/or other medical instrument, which can require the deployment procedure to be repeated. Repeating the deployment procedure can involve repeated accessing of the vessel or body cavity, with its inherent dangers and patient discomfort, and/or the need of a new wire and/or insertion kit.

In the situation where the incision is too long, this can prevent the skin or tissue surrounding the opening from sealing around the catheter or instrument, which can enable bleeding and allow disease-causing agents (e.g., microorganisms, bacteria, viruses) to migrate along an external surface of the catheter into the target body lumen.

Systems, apparatus, and methods described herein can reduce the risk of complications resulting from placement of a catheter or other instrument into a body lumen. As further described below, a cutting device can be designed to consistently form a controlled incision in skin that addresses the aforementioned problems, allowing for placement of the desired catheter or instrument into a body lumen without complications.

Suitable examples of cutting devices are described in P.C.T. Patent Application No. PCT/US2020/031047, titled “Cutting Device and Related Systems and Methods,” filed May 1, 2020, U.S. patent application Ser. No. 16/861,735, filed Apr. 29, 2020, titled “Cutting Device and Related Systems and Methods,” now U.S. Pat. No. 10,813,665, U.S. patent application Ser. No. 16/834,068, filed Mar. 30, 2020, titled “Cutting Device and Related Systems and Methods,” and U.S. patent application Ser. No. 16/402,921, filed May 3, 2019 titled “Cutting Device and Related Systems and Methods,” now U.S. Pat. No. 10,603,071, the disclosure of each of which is incorporated by reference herein.

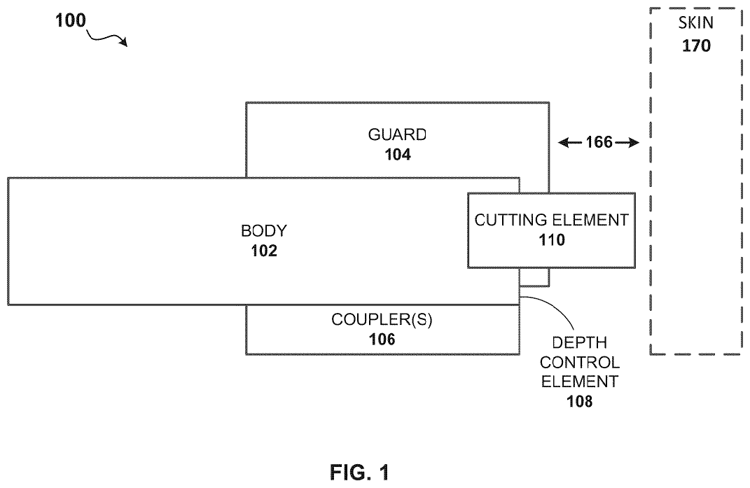

As illustrated schematically in FIG. 1, an example cutting device 100 can include a body 102, a cutting element 110, and a coupler(s) 106. The example cutting device 100 can be placed proximate to a skin 170 of a subject and used to form an incision in the skin 170, e.g., by moving the cutting element 110 through the skin 170. The body 102 can support the cutting element 110 and optionally include a guard 104 designed to expose the cutting element 110 to form the desired incision in the skin 170.

In some embodiments, the body 102 can define a volume, recess, or area for housing the cutting element 110 in a fixed position such that a cutting surface of the cutting element 110 is maintained in a desired position during operation. In some embodiments, the cutting element 110 is coupled to the body 102 via a keying feature, fasteners (e.g., pins, bolts, clips, screws, etc.), adhesive, and/or the like. The guard 104 can be designed to move related to the body 102, e.g., as schematically depicted using arrow 166, to cover or expose the cutting element 110 in a deployed position, such that the cutting element 110 can form an incision in the skin 170. The cutting element 110 can include one or more cutting edges. In some embodiments, the cutting edge is an outer edge that faces away from an instrument.

The guard 104 can include one or more actuation mechanisms for deploying the cutting element 110, e.g., for exposing the cutting element 110, for allowing differential incision lengths, etc. In some embodiments, the guard 104 can include a movable component or actuator (e.g., a slider, button, tab, lever) that can be moved (e.g., slid along a length of the body 102) to deploy the cutting element 110. In some embodiments, the actuator can include a safety mechanism (e.g., tab, etc.) to prevent the cutting element 110 from being exposed undesirably. In some embodiments, the guard 104 can include electrically powered components (e.g., components driven and/or powered by a battery or other power source) for deploying the cutting element 110. In some embodiments, the guard 104 can include components driven mechanically, electrically, magnetically, pneumatically, hydraulically, etc.

In some embodiments, the cutting device 100 may include a shell (e.g., cover, removeable guard, etc.) instead of the guard 104 while still including the body 102 and a cutting element 110 coupled to the body. The shell may be a protective cover over the cutting element 110 that is removed prior to use. The shell may be formed of silicon or other similar material. A cutting device 100 with a shell instead of the guard 104 can be functionally and/or structurally similar to any of the other cutting devices 100 described herein. In some embodiments, the shell is configured to cover the guard 104.

The cutting element 110 can include one or more cutting surfaces or blades that are designed to penetrate through the skin 170 to form an incision. In some embodiments, the cutting element 110 can include more than one cutting surface along one blade. Alternatively or additionally, the cutting element 110 can include other mechanisms, e.g., an electrode, etc., for cutting through the skin 170. The cutting element 110 can be coupled to (e.g., mounted to) body 102 in a fixed or movable relation, e.g., allowing for a variable length incision. In some embodiments, the cutting element 110 can be removably coupled to body 102. For example, the cutting element 110 can be designed to be removed or detached from body 102, such that the cutting element 110 can be replaced after a single or limited number of uses. In some embodiments, the cutting element 110 and/or body 102 can be disposable components that can be disposed of after a single use, e.g., after being used to provide access to a target site in the subject. In some embodiments, the cutting element 110 extends distally beyond a distal end of the instrument and at an angle toward a guidewire disposed in the instrument. In some embodiments, the cutting element 110 includes an indicator (e.g., line, arrow, marking, etc.) indicating where a distal end of the instrument should be when coupled to the cutting device 100.

The body 102 can be ergonomically shaped such that a user (e.g., a physician) can hold (e.g., grip) the body 102 in single hand. Optionally, if the guard 104 is present, the guard 104 can be positioned on or about the body 102 such that the user can retract the guard 104 while maintaining his hold (e.g., grip) on the body 102 with a single hand. For example, the guard 104 can be a slide that can be moved, e.g., using a thumb of a user, to expose the cutting element 110 during a medical procedure. The body 102 and cutting element 110 can be formed from lightweight material such that a user can comfortably hold the body 102 and cutting element 110 without feeling additional strain during a medical procedure.

The cutting device 100 can include a depth control element 108 that is integrated into and/or coupled to the body 102 and/or cutting element 110. The depth control element 108 can be designed to limit a depth of penetration or insertion of the cutting element 110 into the skin 170 so as to control a size of the incision formed by the cutting element 110 in the skin 170. In some embodiments, the depth control element 108 can be designed to contact a surface of the skin 170 to control a depth of insertion of the cutting element 110. For example, the depth control element 108 can include a surface, protrusion, or other physical structure that can be disposed at a point along the cutting element 110 and/or body 102 such that it would contact a surface of the skin 170 when the cutting element 110 has been inserted a predetermined depth into the skin 170. In some embodiments, the depth control element 108 can include one or more components that restrict movement of the cutting element 110, thereby controlling a depth of insertion of the cutting element 110. For example, the depth control element 108 can include one or more protrusions and/or surfaces that interfere (e.g., lock) with one another to prevent further movement of the cutting element 110 in a direction toward the skin 170 after the cutting element 110 has penetrated a predetermined depth into the skin. In some embodiments, the depth control element 108 can include one or more sensor(s) (e.g., a light sensor, a pressure sensor, etc.) that can be designed to sense or detect a depth of insertion. In some embodiments, the depth of insertion is less than about 20 mm.

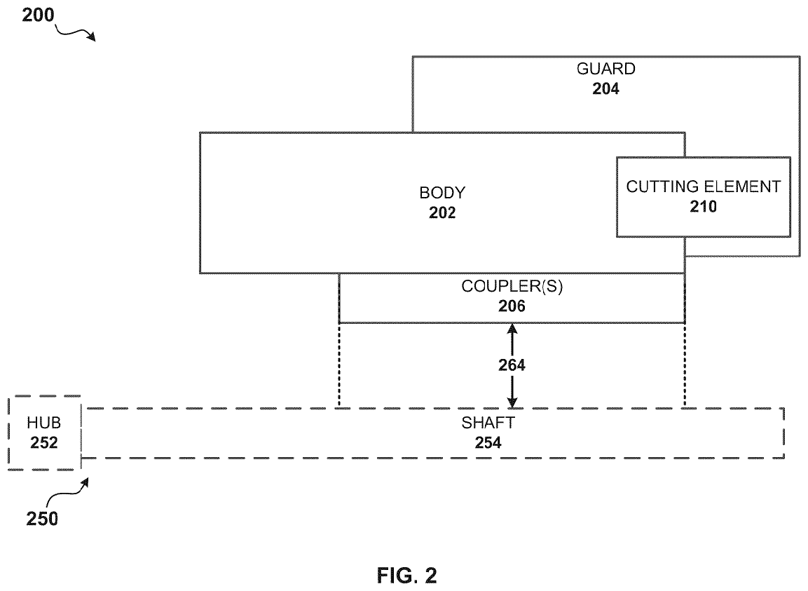

The coupler(s) 106 can include one or more components designed to couple the body 102 (or other components of the cutting device 100) to an instrument (e.g., a dilator, a catheter, etc.). In some embodiments, the coupler(s) 106 can be designed to reversibly couple the body 102 to the instrument. For example, the coupler(s) 106 can be designed to couple and decouple the body 102 to the instrument and/or additional instruments, as many times as needed without comprising its structure. In some embodiments, the coupler(s) 106 can include mechanical components, e.g., a clasp, a clip, etc. that can attach around the instrument to couple the body 102 to the instrument. The mechanical component can be designed to be flexible such that it can bend to fit around (e.g., grip onto) the instrument. For example, the coupler(s) 106 can be resiliently deformable. In some embodiments, the mechanical component can include multiple staggered mechanical components. Alternatively or additionally, the mechanical component can be designed to be mechanically biased (e.g., with a spring) or electrically driven to change between different configurations for attaching to and/or detaching from the instrument. The coupler(s) 106 can be configured to maintain the coupling between the body 102 and the instrument by interference fit, press fit, friction fit, and the like. In some embodiments, the coupler(s) 106 can be configured to allow coaxial movement of the instrument such that the body 102 (and other components of the cutting device 100) can slide or move along a length of the instrument. The coupler(s) 106 can be integrated into or attached to the body 102. As further described with reference to FIG. 2, the coupler(s) 106 can be configured to attach the body 102 (or other components of the cutting device 100) to the instrument such that the cutting element 110 is positioned to create an incision in the skin 170 for receiving the desired instrument. In some embodiments, the coupler(s) 106 can be sized to a specific size of instrument. Alternatively, the coupler(s) 106 can be configured to couple to instruments within a range of sizes, e.g., such as with adjustable components (e.g., an adjustable clamp or a deformable plug that accommodates a range of sizes). In some embodiments, the coupler(s) 106 can include at least three couplers.

In some embodiments, the cutting device 100 can include a component (e.g., a protrusion) that can assist with positioning the cutting device 100 along a length of the instrument. For example, the cutting device 100 can include a beam, rod, or other like structure that can be configured to extend from a proximal end of the body 102 to a proximal end or portion of the instrument, such that when the cutting device 100 is attached to the instrument (e.g., via the coupler(s) 106), such structure indicates the location at which the cutting device 100 should be positioned relative to the proximal or distal end of the instrument.

The positioning of a cutting device relative to an instrument can be further understood with reference to FIG. 2. As illustrated schematically in FIG. 2, an example cutting device 200 is shown in relation to an example instrument 250 (e.g., a surgical instrument) with which the cutting device 200 can be used. The cutting device 200 can be structurally and/or functionally similar to the cutting device 100. For example, the cutting device 200 can include a body 202, a guard 204, a cutting element 210, and a coupler(s) 206. The instrument 250 can include a hub 252 and a shaft 254. The cutting device 200 can be reversibly attachable to and/or supportable by the instrument 250, e.g., via the coupler(s) 206 attaching to the shaft 254 (e.g., dilator, etc.) of the instrument 250, as represented by line 264. The shaft 254 of the instrument 250 can include portions that are rigid and/or flexible. FIG. 2 further shows the guard 204 covering the cutting element 210, but the guard 204 can be retracted to expose the cutting element 210.

The cutting device 200 can be used to form a skin incision in skin (e.g., skin 170) for receiving the instrument 250, e.g., such that the instrument 250 can be received into a target body lumen (e.g., a blood vessel). The instrument 250 can be, for example, a dilator, a catheter, a chest tube, or the like. In an embodiment, the instrument 250 can be a dilator that has a distal end configured to be inserted through the incision and a proximal end including a hub 252. The dilator can have a tapered distal end that facilitates insertion into and subsequent dilation of tissue and/or blood vessels deep to the level of the skin incision (e.g., deeper than a level of the skin incision in skin 170). In some embodiments, the dilator can range in size from about 3 French to about 12 French (i.e., about 1 mm to about 4 mm), including all values and subranges in between. The instrument 250 can include a lumen that extends throughout a length of the instrument 250, i.e., through a length of the hub 252 and a length of the shaft 254. The lumen can be configured to receive a guidewire, e.g., for steering or guiding the instrument 250 into the target body lumen.

In use, the cutting device 200 can be coupled to the instrument 250 such that the cutting element 210 can be configured to form an incision for receiving the instrument 250, e.g., as part of the Seldinger technique. For example, a needle can be used to create a puncture site in tissue (e.g., skin 170), and a guidewire can be inserted through the needle into a body lumen (e.g., a blood vessel) deep to the insertion site at the skin level. The needle can be removed, and the instrument 250 (e.g., a dilator) with the body 202 can be slid over the guidewire until a distal end of the instrument 250 contacts a surface of the skin or if within a predetermined distance to the skin. The cutting element 210 can then be exposed to form an incision in the skin that extends from the puncture site and is sized to receive the instrument 250. The body 202 can be attached to the instrument 250 at a specific location that enables the cutting element 210, when actuated, to form such an incision. Once the incision is formed, the body 202 can be removed (e.g., detached, decoupled) from the instrument 250, and the instrument 250 can be inserted through the incision into the body (e.g., and into a lumen or cavity). If the instrument 250 is a dilator (or dilators), the dilator (or dilators) can be used to dilate the tissue and/or blood vessels deep to the incision, and be subsequently withdrawn over the guidewire allowing the appropriately sized catheter (or other instrument) to be guided down the guidewire and placed in the body lumen.

In some embodiments, where the instrument 250 includes a tapered distal end, e.g., such as a dilator with a tapered distal end, the cutting element 210 can be supported such that it is angled toward the puncture site (e.g., angled to follow the taper of the instrument 250), such that the cutting element 210 can form an incision that extends from the puncture site, e.g., preventing any possibility of leaving a skin bridge as described in FIGS. 5A-5B.

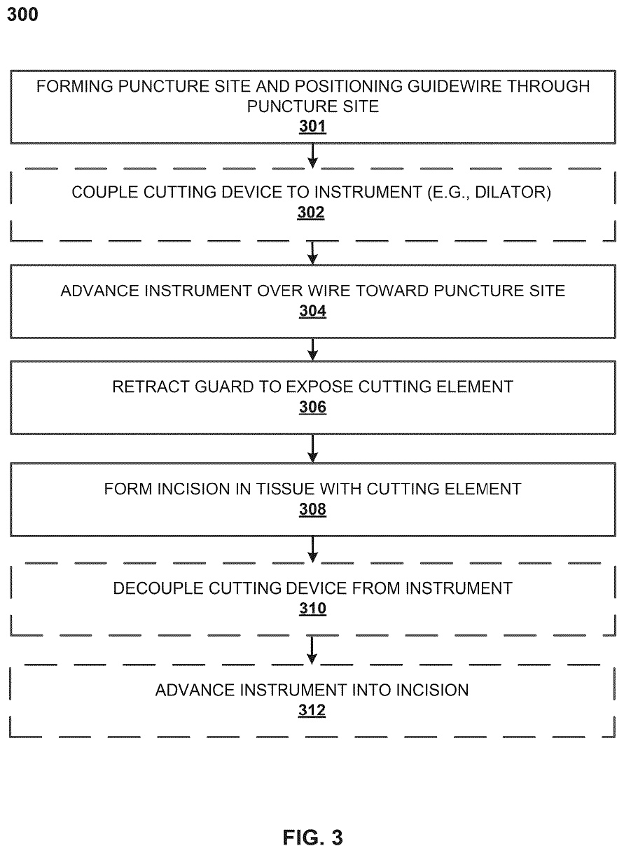

Referring now to FIG. 3, a method 300 of performing a medical procedure on a subject using a cutting device, such as any of those described herein (e.g., cutting device 100, 200, 600, 700, and/or 1200), is shown and described.

The method 300 can be a method of forming an incision for receiving an instrument (e.g., instrument 250). The instrument can be, for example, a dilator. The method 300 can include forming, e.g., using a needle, a puncture site in tissue (e.g., skin 170), and advancing a guidewire through the needle into a body lumen, at 301. The needle can be removed, leaving the guidewire positioned traversing the puncture site. The instrument can define a lumen configured to receive the guidewire. The method 300 can optionally include coupling a cutting device to the instrument, at 302. In some embodiments, a positioning element can also be coupled to the instrument to assist with appropriate positioning of the cutting device on the instrument and to prevent proximal movement of the cutting device relative to the instrument. In some embodiments, a portion of the cutting device (e.g., a guard) can include an indicator marking indicating a desirable position of the instrument. In some embodiments, the cutting device can be reversibly coupled to the instrument such that the cutting device can be removed (e.g., detached, decoupled) from the instrument, e.g., when moved in a direction radially away from the longitudinal axis (e.g., a central longitudinal axis) of the dilator. In some embodiments, the method 300 may not include coupling the cutting device to the instrument because the instrument may be provided (e.g., packaged, sold, etc.) with the cutting device pre-attached, to, but removable or detachable from, the instrument.

At 304, the method 300 can include advancing the instrument over and along the guidewire toward the puncture site, e.g., until a distal end of the instrument is against a surface of the tissue. At 306, the method 300 optionally includes retracting a guard (e.g., guard 104) to expose a cutting element (e.g., 110, 210) of the cutting device. As described above with reference to FIG. 1, retracting the guard can include moving (e.g., sliding) the guide to expose the cutting element. For example, the cutting element can be exposed by moving a slider of the cutting device in a proximal direction, e.g., using a thumb of a user when the slider is disposed on a side of the cutting device accessible by the thumb. When the cutting element has been exposed (e.g., deployed) with the guard retracted, the cutting element can be configured to form an incision in the tissue. In some embodiments, the method 300 may include removing a guard, e.g., a cutting element cover. In such embodiments, the cutting device may include a cap or other component that may shield the cutting element during transport but be removed from the cutting element prior to use of the cutting device.

The cutting element, after being actuated or exposed by retracting a guard, can form an incision in the tissue, at 308. In some embodiments, the cutting device and the dilator can be advanced toward the tissue after the cutting element has been extended to form the incision. The incision can be formed such that it extends from the puncture site and is sized to receive the instrument. The incision, for example, can be formed to have a length that is substantially equal to half the perimeter of the instrument. In some embodiments, the cutting device includes a depth control element (e.g., depth control element 108), which can control a distance that the cutting element can extend beyond a distal end of the cutting device and/or a distance that the cutting element can be inserted into the tissue, thereby controlling a depth of the incision. For example, a surface of the cutting device (e.g., a surface of a housing of the cutting device) can be configured to contact the tissue once the cutting element has been inserted a set distance into the tissue. Alternatively or additionally, one or more components of the cutting device may lock to prevent further extension of the cutting element beyond a distal end of the cutting device. In some embodiments, the cutting element can be movable, after forming the incision, from its extended position back to its retracted position.

At 310, the method 300 optionally includes removing (e.g., decoupling, detaching) the cutting device from the instrument, e.g., by moving the cutting device in a direction radial to the longitudinal axis of a body (e.g., shaft 254) of the instrument.

At 312, the method 300 optionally includes, after forming the incision, advancing the instrument over the guidewire into the incision. In some embodiments, the instrument is advanced until a distal end of the instrument is disposed within the body lumen. In some embodiments, when the instrument is a dilator, the instrument can be removed from the incision, and subsequent, larger instruments can be used to further dilate the subdermal tract to a final dilatation that allows a final catheter to be slid over the guidewire into the body lumen. The catheter can then provide access to the body lumen, e.g., via a lumen of the catheter.

In an example embodiment, a method can include disposing a dilator over a wire that has been inserted into a puncture site formed in tissue, the dilator including a proximal hub and an elongate body, the elongate body of the dilator having a cutting device reversibly coupled thereto; advancing the dilator with the cutting device toward the puncture site; moving a guard to transition a cutting element of the cutting device from (1) a fully covered position in which the cutting element is covered by the guard to (2) a fully exposed position in which a distal portion of the cutting element extends distally from the housing to a distal end of the dilator; inserting the distal end of the dilator and the cutting element into the tissue such that the cutting element forms an incision extending from the puncture site; and moving the cutting device radially away from a longitudinal axis of the dilator while maintaining the dilator over the wire to decouple the cutting device from the elongate body of the dilator. The distal end of the dilator and the cutting element can be inserted into the tissue until a distal surface of the housing contacts the tissue and prevents further insertion of the cutting element into the tissue so that the incision has a length that is approximately equal to a half the perimeter of a catheter to be placed through the incision. The guard can be used to expose the cutting element after the distal end of the dilator is positioned against the puncture site, or the cutting element can be exposed while the dilator with the cutting device is advanced toward the puncture site. The cutting element can be disposed radially outward from a lumen of the dilator in the fully retracted and fully extended positions when the cutting device is coupled to the elongate body of the dilator. The cutting element can be exposed in response to moving an actuation mechanism of the guard in a proximal direction. The method can also include moving, after forming the incision, the guard to cover the cutting element, the cutting device being decoupled from the dilator after the cutting element is moved back to the fully retracted position. The method can also include advancing the dilator after decoupling the cutting device from the dilator to dilate the incision; removing the dilator from over the wire; and advancing the catheter over the wire and into the incision. The method can also include moving, after forming the incision, a spacer laterally away from the longitudinal axis of the dilator while maintaining the dilator over the wire to decouple the spacer from the elongate body of the dilator. The spacer can be reversibly coupled to the elongate body of the dilator and be configured to prevent proximal movement of the cutting device relative to the dilator.

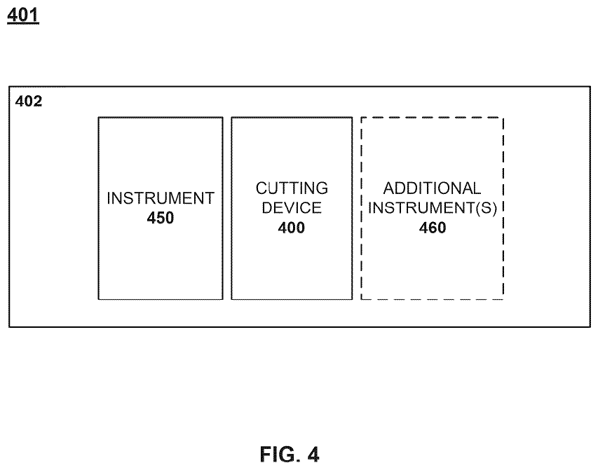

Referring now to FIG. 4, a schematic diagram depicting components of a surgical system is shown and described. The components depicted in FIG. 4 can be provided in a kit 401, which can be provided to a physician for use during a surgical procedure. The kit 401 can be provided in packaging 402, such as, for example, a box, a bag, etc. The packaging 402 can be configured to keep the components of the surgical system sterile prior to use. The surgical system can include an instrument 450 and a cutting device 400, and optionally and/or additional instrument(s) 460.

The cutting device 400 can be functionally and/or structurally similar to other cutting device described herein (e.g., cutting device 100, 200). For example, in some embodiments, the cutting device can include a cutting element (e.g. cutting element 110, 210) configured to form an incision in tissue.

When included together in packaging 402, the cutting device 400 can be pre-coupled to the instrument 450 at a desired location, e.g., allowing the creation of an incision in tissue. For example, the cutting device 400 can be coupled to the instrument such that a cutting element (e.g., cutting element 110, 210) of cutting device 400 can be configured to form an incision for receiving the instrument 450, e.g., an incision that extends from a puncture site and is sized for receiving the instrument 450. Alternatively, the cutting device 400 and the instrument 450 can be separately placed within the packaging 402, e.g., within separate inner compartments and/or containers.

One or more components of kit 401 can be configured to be disposed after a single use. For example, the cutting device 400 can be configured for one-time use, e.g., the cutting device 400 can include one or more components that lock after a single use.

The instrument 450 can be a dilator, a catheter, a chest tube, or other surgical instrument. The instrument 450, similar to instrument 250 described above, can define a lumen configured to receive a wire such as a guidewire that extends or is otherwise extendable through a puncture site. In some embodiments, the kit 401 can optionally include an appropriately sized guidewire (not shown) for use with the instrument 450. The instrument 450 can include a hub (e.g., similar to hub 252) at a proximal end and body (e.g., similar to shaft 254). The incision formed by the cutting device 400 can be sized to receive the instrument 450. For example, the incision can have a length that is substantially equal to a diameter of the instrument 450. In some embodiments, the kit 401 can include the instrument 450 having a first diameter, and additional instruments 460 having diameters different than the first diameter. For example, the instrument 450 can be a dilator with a first dimeter, and an additional instrument 460 can be a dilator having a diameter greater than the first diameter of the dilator. In such embodiments, the kit 401 can contain a set of dilators that progressively increase in size, e.g., a progressive set of dilators. The dilators can increase in size from (1) a first diameter of the dilator that is configured to couple to the cutting device 400 to (2) a second diameter equivalent to a diameter of the final instrument (e.g., catheter) to be inserted into the incision and into a target vessel. Each of the dilators can be used to dilate the incision to the size of the surgical instrument. In these instances, the cutting device can create an incisional length equal to half the perimeter of the final dilator/catheter/tube to be inserted.

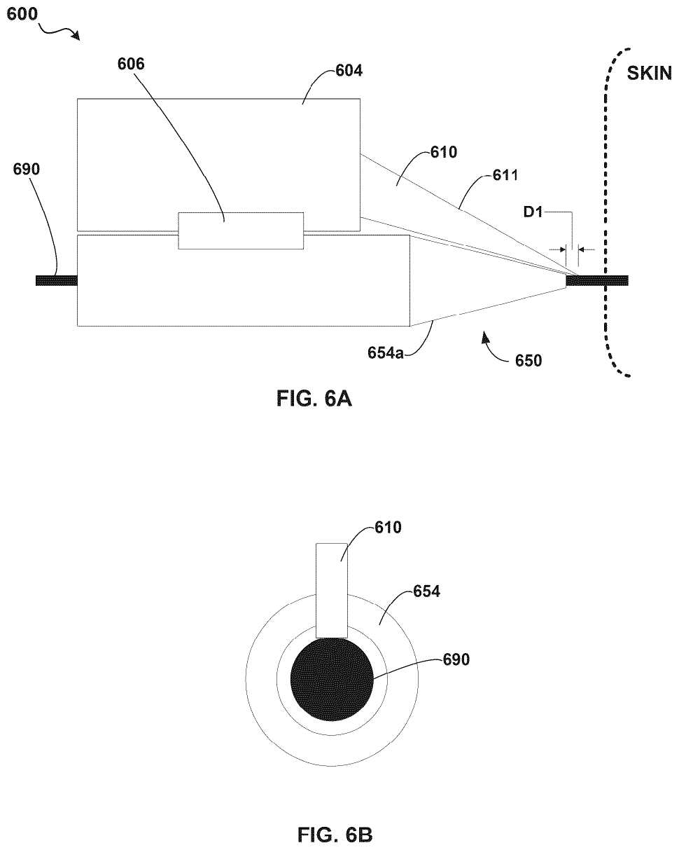

Referring now to FIGS. 6A-6B, views of another example cutting device 600 are shown and described. The cutting device 600 is configured to form an incision without a skin bridge as described in reference to FIGS. 5A-5B. FIG. 6A schematically illustrates a distal end of the cutting device 600 and the instrument 650. And FIG. 6B schematically illustrates a cross-sectional view of a distal end portion of the cutting device 600 and the instrument 650. The cutting device 600 can include components that are structurally and/or functionally similar to other cutting devices described herein. For example, the cutting device 600 can include a guard 604, a cutting element 610, and a couplers 606. The cutting device 600 can be used with the instrument 650, similar to other instruments described herein.

The instrument 650 can be implemented as a dilator having a shaft 654 that includes a tapered distal end 654a. The instrument 650 can define a lumen configured to receive a wire such as guidewire 690 that extends or is otherwise extendable through a puncture site. The cutting device 600 can be configured to reversibly couple to the instrument 650, as described herein. In some embodiments, the cutting device 600 may be designed to move or slide longitudinally along a shaft 654 of the instrument 650.

A body of the cutting device 600 can support the cutting element 610. For example, the body can include a clamp, protuberance, knob, bump, or other attachment mechanism for holding and supporting the cutting element 610. The cutting element 610 can include an inner edge (not depicted) and an outer cutting edge 611. In some embodiments, the outer cutting edge 611 can include multiple distinct portions configured to cut tissue. The guard 604 can be translated along the body as to expose the cutting element 610. The cutting element 610 is configured such that a distal tip of the cutting element 610 extends a distance D1 past the distal tip of the shaft 654. In some embodiments, the distance D1 is between about 0.5 mm and about 3 mm, inclusive of all ranges and values therebetween. In some embodiments, the distance D1 is less than about 3 mm, less than about 2 mm, or less than about 1 mm.

As depicted in FIGS. 6A and 6B, a portion of the cutting element 610 (e.g., the distal tip of the cutting element 610) extends into a space define by the inner lumen of the instrument 650 such that the cutting element 610 abuts or is near the wire 690. The cutting element 610 can then engage the skin in a distal direction to form an incision that extends from a puncture site in which the wire 690 is disposed. By enabling the cutting element 610 to extend into the space defined by the inner lumen of the instrument 650 to the wire 690, the formation of a skin bridge is prevented. An inner edge of the cutting element 610 is configured to substantially follow the shape of the tapered distal end 654a.

The coupler(s) 606 can reversibly couple the body to the instrument 650. For example, the coupler(s) 606 can include a flexible component such as a clip or clasp that is designed to removably couple the body 602 to the instrument 650 such as by interference fit, press fit, friction fit, and the like. The coupler(s) 606 can be integrated into or otherwise used in conjunction with the body 602 so as to enable and facilitate reversible coupling of the cutting device 600 to the instrument 650, such as described herein. In some embodiments, for example, the coupler(s) 606 can include two or more attachment points to prevent movement of the cutting device 600 with respect to the instrument 650 due to torsional forces.

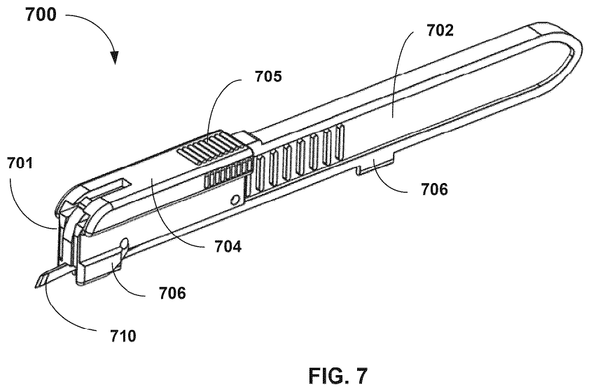

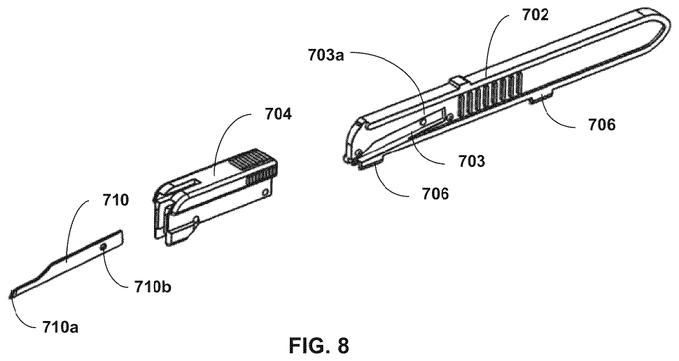

FIGS. 7-11 depict yet another example of a cutting device 700, according to embodiments. The cutting device 700 can include components that are structurally and/or functionally similar to other cutting devices described herein, e.g., cutting device 100, 200, and 600, and includes a stationary cutting element 710 and a movable guard 704. For example, the cutting device 700 can include a housing 702, an actuation mechanism implemented as a guard or shield 704, a cutting element 710, one or more coupling coupler(s) 706, and a depth control element implemented as a tissue contact surface 701.

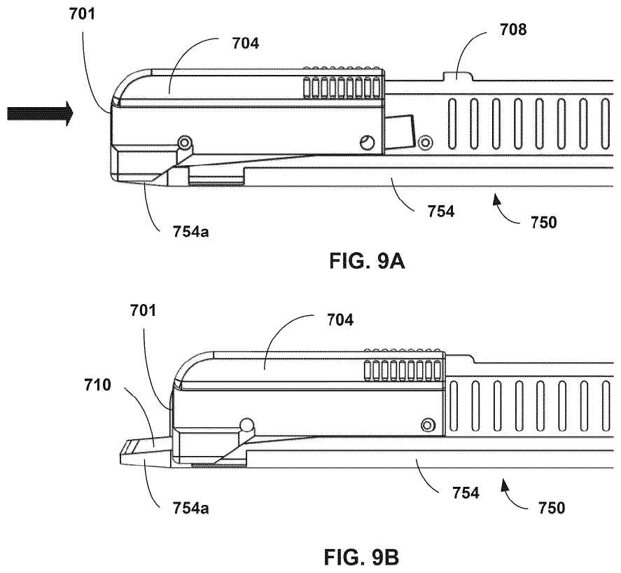

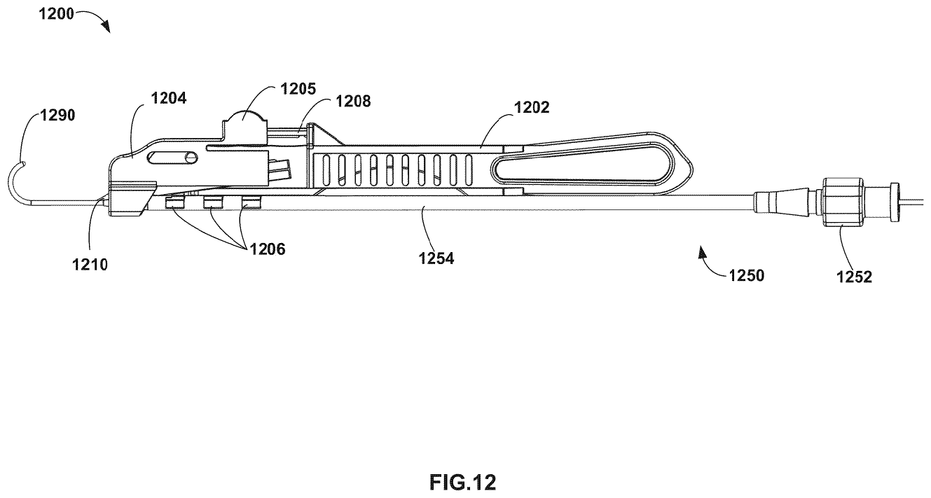

The cutting device 700 is reversibly coupleable to a medical device, e.g., a dilator 750 as shown in FIGS. 9A-11, via the coupler(s) 706. The coupler(s) 706 includes two fastening or coupling points. At each coupling point, one or more plugs or gripping mechanisms (which can be rigid or flexible) can be used to reversibly couple to the dilator 750. Alternatively or additionally, other types of coupling elements, e.g., clamps, clips, magnets, etc., can also be used to grip the dilator 750. The coupler(s) 706 can be configured to couple to the dilator 750 from a radial direction, e.g., by moving the cutting device 700 in a direction toward the longitudinal axis of the dilator 750, and similarly to decouple from the dilator 750 in a radial direction. As shown in FIG. 11, the dilator 750 can include a proximal end including a hub 752 (e.g., as described with reference to instrument 250 depicted in FIG. 2). Accordingly, the cutting device 700, by being radially coupleable and decouplable from the dilator 750, is capable of being separated from the dilator 750 even while the dilator 750 is disposed over a guidewire 790 that is disposed within a patient. Once coupled to the dilator 750, the coupler(s) 706 can generate sufficient friction against the dilator 750 to prevent movement (e.g., sliding and/or rotation) of the cutting device 700 relative to the dilator 750. The coupler(s) 706 includes more than one coupling point such that the coupling points collectively prevent pivoting and/or displacement of the cutting device 700 relative to the dilator 750. The dilator 750 itself may have a degree of flexibility, and therefore having multiple points of coupling can ensure that the dilator 750 remains aligned with the cutting device 700 during use.

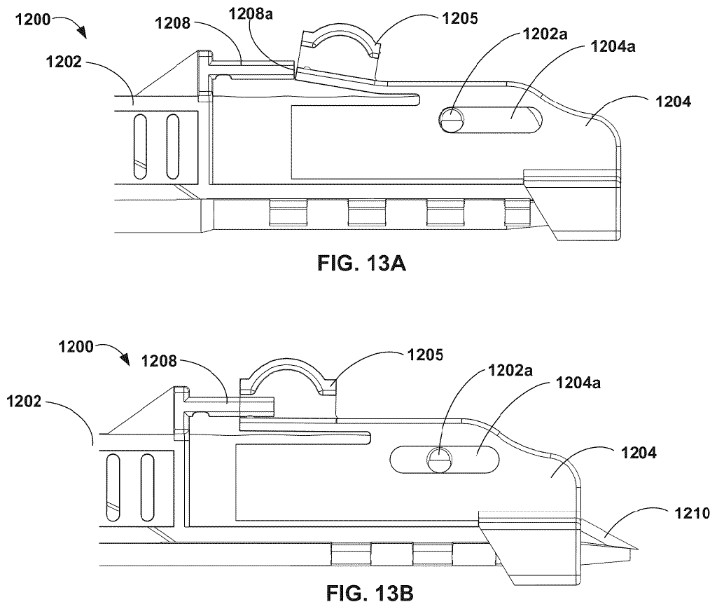

In the embodiment depicted, the coupling point can be located at or near a distal or front end of the cutting device 700, to further ensure alignment between a cutting element 710 of the cutting device and a longitudinal axis of the dilator (e.g., by reducing the risk of displacement between the end of the dilator 750 and the cutting device 700). Such alignment ensures that the cutting device 700 does not form an incision that is offset from the puncture site, as depicted in FIG. 5A. In some embodiments, one or more components of the coupler(s) 706 can be controlled, e.g., via a mechanical or electrical actuator, to open and/or close to couple and decouple the cutting device 700 to the dilator 750. While two coupling points are depicted in the figures, it can be appreciated that any number of coupling points (as seen and described in reference to FIGS. 12-15) or a continuous coupling point can be used to couple the cutting device 700 to a medical instrument such as dilator 750.

The housing 702 can be configured to support the cutting element 710 of the cutting device 700. In the example embodiment depicted, the housing 702 can include a recess 703 for receiving the cutting element 710 and supporting it in a fixed position. The recess 703 can include a feature 703a (e.g., a protrusion) that can engage with an opening 710b in the cutting element 710 to hold it in place relative to the housing 702. While a recess and protrusion are depicted as mechanisms for holding the cutting element 710 in a fixed position relative to the housing 702, it can be appreciated that other mechanisms (e.g., mechanical such as via a screw, fastener, etc., magnetic via one or more magnets, and/or adhesive) can be used to hold the cutting element 710 in a fixed position relative to the housing 702.

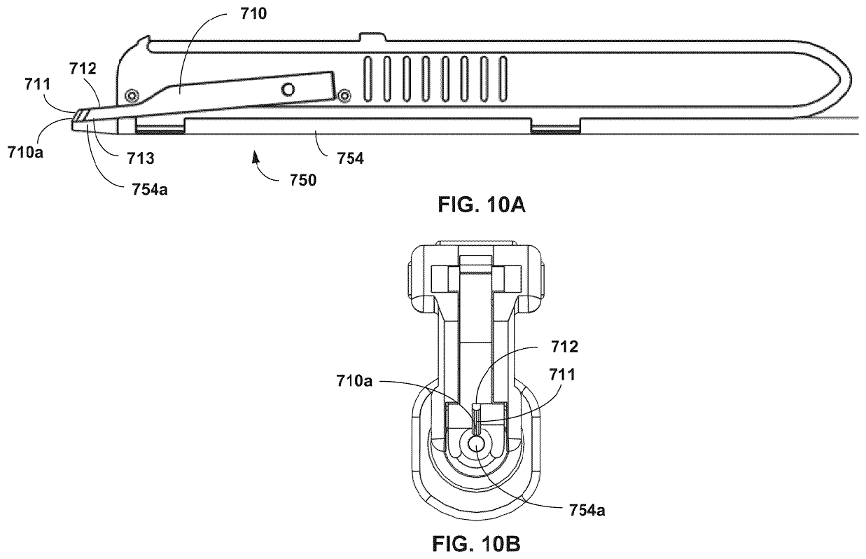

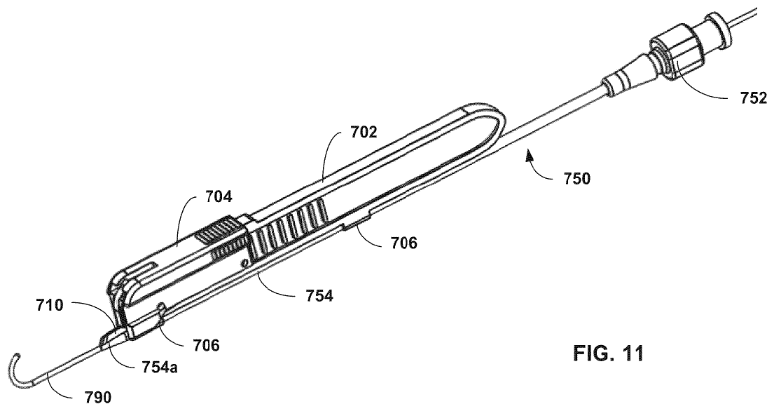

The housing 702 can be configured to support the cutting element 710 in a fixed position such that the cutting element 710 is angled toward a longitudinal axis of the dilator 750. In particular, the cutting element 710 can be coupled to the dilator 750 such that a distal end 710a of the cutting element 710 is aligned with a distal end 754a of a body 754 of the dilator 750. The cutting element 710 can include an inner edge 713 and one or more outer edges 711, 712. When the cutting device 700 is couped to the dilator 750, the inner edge 713 can extend along or substantially along the distal end 754a of the body 754 in contact with an outer surface of the distal end 750a, as depicted in FIG. 10A. In other words, the cutting element 710 can be angled such that its inner edge 713 extends along the outer surface of the distal end 754a of the body 754. The cutting element 710, by being disposed against an outer surface of the dilator 750 and with its distal end 710a extending past the distal end 754a of the dilator 750, ensures that the incision formed by the cutting element 710 extends from the puncture site that the dilator 750 is being inserted through. In some embodiments, the distance the distal end 710a extends past the distal end 754a is less than about 5 mm. In some embodiments, the distance the distal end 710a extends past the distal end 754a is between about 0.5 mm and about 5 mm, inclusive of all ranges and values therebetween.

In some embodiments, each of the edges 711, 712, 713 can be cutting edges, e.g., designed to cut tissue. In some embodiments, the outer edge 711 can be a cutting edge while the inner edge 713 and the outer edge 712 can be non-cutting edges. In some embodiments, a subset of the inner and outer edges 711, 712, 713 can be cutting edges (or have portions that are cutting portions). In some embodiments, the cutting element 710 can be moveably supported by the housing 702. For example, the cutting element 710 can be movable or actuatable relative to the housing 702. In such embodiments, the cutting element 710 may be moved, e.g., translated, rotated, pivoted, etc., to change the incision that is formed. For example, the cutting element 710 may be pivoted or rotated about an axis to increase the size of an incision that is formed. As the cutting element 710 is pivoted, its outer and/or inner cutting edges 711, 712, 713 can be configured to cut the tissue to form a larger incision. In some embodiments, a spring similar to spring can also be used to apply a force upon the cutting element 710, e.g., to push the cutting element 710 against an outer surface of the dilator 750.

The housing 702 can also be configured to support the guard 704. The housing 702 can support the guard 704 in a location where it can be easily manipulated by a digit of a user (e.g., an index finger or a thumb of a user). For example, as depicted in FIG. 7, the housing 702 can support the guard 704 on a side of the cutting device 700 that is opposite from the side including the coupler(s) 706. The guard 704, as disposed, can be capable of being operated by both left and right handed users. The guard 704 can be moved between a first configuration (i.e., a fully extended position), as depicted in FIG. 9A, and a second configuration (a fully retracted position), as depicted in FIG. 9B. When the guard 704 is in the first configuration, it covers the cutting element 710, e.g., such that any cutting surfaces or edges of the cutting element 710 are shielded within the guard 704. When the guard 704 is in the second configuration, the cutting element 710 is exposed, e.g., such that the cutting element 710 can form the incision. In the second configuration, the guard 704 also provides a tissue contacting surface 701, which can be configured to contact the tissue surface near the puncture site to prevent further insertion of the cutting element 710 into the tissue, e.g., to control the size of the incision. A stop 708 coupled to or integrated with the housing 702 can be configured to stop the retraction of the guard 704 and to define the proper positioning of the guard for controlling the depth of insertion of the cutting element 710. The guard 704 can be moved between the first configuration and second configurations by a user manually retracting the guard 704. Alternatively, the guard 704 can be moved between the first and second configurations via one or more other mechanisms, e.g., electronically, magnetically, etc.

The fixed arrangement of the cutting element 710 relative to the housing 702, and therefore to the dilator 750 coupled to the housing 702, can provide certain advantages. For example, when coupling the cutting device 700 to the dilator 750, the guard 704 and the cutting element 710 can be configured to terminate at or about the same longitudinal location (e.g., as depicted in FIG. 9A), which can enable a user can visually align the distal end of the guard 704 (i.e., surface 701) and the cutting element 710 to ensure proper positioning of the cutting device 700 relative to the dilator 750. Once coupled, the guard 704 can be retracted, e.g., to the position shown in FIG. 9B, and a user can slide dilator 750 with cutting element 700 coupled thereto down the length of the guidewire 790 to form the incision in the tissue. When the guard 704 is retracted, it does not move the cutting element 710, which can ensure that none of the cutting portions or edges of the cutting element 710 may inadvertently cut any nearby structures (e.g., the dilator 750). The fixed position of the cutting element 710 relative to the dilator 750 also ensures that the cutting element 710 always remains aligns with the distal end of the dilator 750, which further ensures that an accurately sized and positioned incision is formed.

FIGS. 12-15 depict yet another example of a cutting device 1200, according to embodiments. The cutting device 1200 can include components that are structurally and/or functionally similar to other cutting devices described herein, e.g., cutting device 100, 200, 600, and 700, and includes a stationary cutting element 1210, a movable guard 1204, and a staggered couplers 1206. For example, the cutting device 1200 can include a body 1202, the guard 1204, a cutting element 1210, one or more couplers 1206, and a depth control element implemented as a tissue contact surface 1201. While not depicted, cutting device 1200 can be used with a positioning element but can also be used alone.

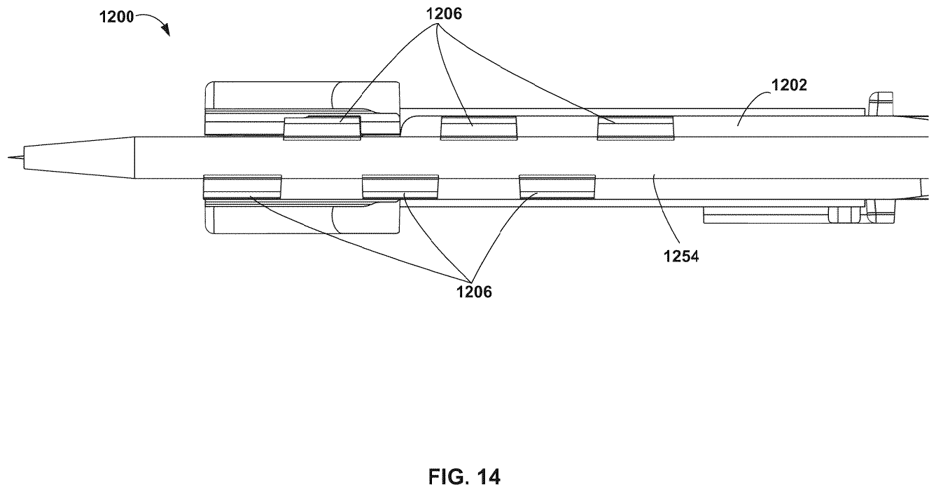

The cutting device 1200 is reversibly coupleable to a medical device, e.g., a dilator 1250, via the couplers 1206. The couplers 1206 include several staggered fastening or coupling points as seen in FIG. 14. Each of the coupling points can be functionally and/or structurally similar to the coupling points described herein, e.g., the coupler(s) 706 in FIGS. 7-12. In some embodiments, such as the embodiment seen in FIG. 14, the couplers 1206 include seven coupling points, with four coupling points on one side of the body 1206 and three coupling points on the opposite side of the body 1206. The couplers 1206 include legs that are configured to extend around one side of the dilator 1250 with legs of adjacent coupler 1206 being offset from one another.

The staggered coupling points collectively prevent pivoting and/or displacement of the cutting device 1200 relative to the dilator 1250. The staggered coupling points can also provide for easier coupling of the cutting device 1200 to the dilator 1250. The staggered coupling points are located at or near the distal end or front end of the cutting device 1200 to ensure alignment between the cutting element 1210 of the cutting device and a longitudinal axis of the dilator (e.g., by reducing the risk of displacement between the end of the dilator 750 and the cutting device 700). Such alignment ensures that the cutting device 1200 does not form an incision that is offset from the puncture site, as depicted in FIG. 5A. While seven coupling points are depicted in the figures, it can be appreciated that any number of coupling points (as seen and described in reference to FIGS. 12-15) or a continuous coupling point can be used to couple the cutting device 1200 to a medical instrument such as dilator 1250.

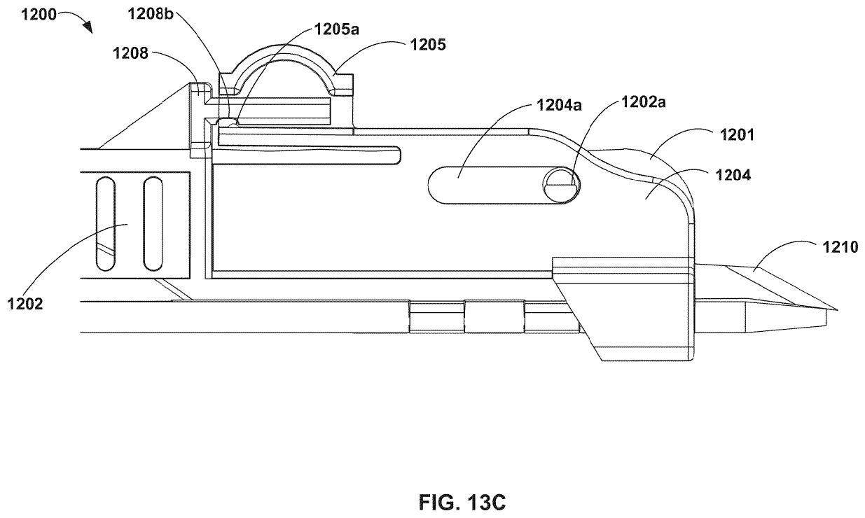

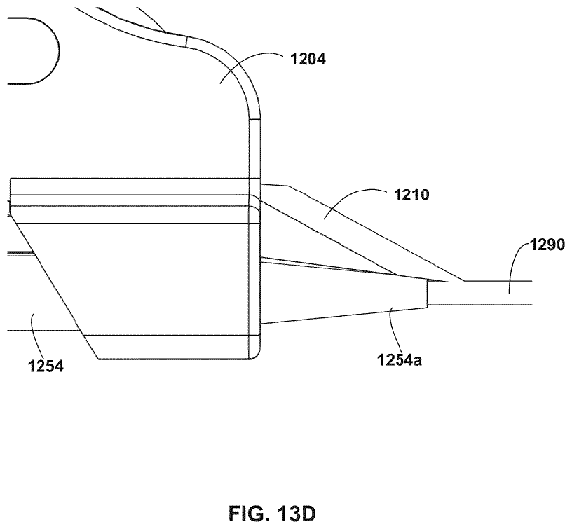

The body 1202 can be configured to support the cutting element 1210 of the cutting device 1200. In the example embodiment depicted, the housing 702 can receives the cutting element 1210 and supports the cutting element 1210 in a fixed position. The body 1202 can include a recess or similar features as described in reference to FIGS. 7-11. The body 1202 can be configured to support the cutting element 1210 in a fixed position such that the cutting element 1210 is angled toward a longitudinal axis of the dilator 1250, as seen in FIG. 13D. In particular, the cutting device 1200 can be coupled to the dilator 1250 such that a distal end 1210a of the cutting element 1210 is aligned with a distal end 1254a of a shaft 1254 of the dilator 1250. As described in reference to FIGS. 6A-6B, the cutting element 1210 extends past the distal end of the distal end 1254a such that the cutting element 1210 abuts or nearly abuts the wire 1290. The cutting element 1210, by being disposed against the wire 1290, ensures that the incision formed by the cutting element 1210 extends from the puncture site that the dilator 1250 is being inserted through and prevents a skin bridge.

In some embodiments, the cutting element 1210 can be moveably supported by the body 1202. For example, the cutting element 1210 can be movable or actuatable relative to the body 1202. In such embodiments, the cutting element 1210 may be moved, e.g., translated, rotated, pivoted, etc., to change the incision that is formed. For example, the cutting element 1210 may be pivoted or rotated about an axis to increase the size of an incision that is formed. As the cutting element 1210 is pivoted, its outer and/or inner cutting edges can be configured to cut the tissue to form a larger incision.

The guard 1204 is configured to translate along the body 1202 to allow for the guard 1204 to shield and/or expose the cutting element 1210. A slot 1204a of the guard 1204 acts as a guide for a protrusion 1202a of the body 1202 as it translates between a extended position (e.g., cutting element 1210 hidden) as seen in FIG. 13A and an retracted position (e.g., cutting element 1210 exposed) as seen in FIG. 13C. The guard 704, as disposed, can be capable of being operated by both left and right handed users. When the guard 704 is in the closed configuration, it covers the cutting element 710, e.g., such that any cutting surfaces or edges of the cutting element 710 are shielded within the guard 704. When the guard 704 is in the open configuration, the cutting element 710 is exposed, e.g., such that the cutting element 710 can form the incision. In the open configuration, the guard 1204 also provides a tissue contacting surface 1201, which can be configured to contact the tissue surface near the puncture site to prevent further insertion of the cutting element 1210 into the tissue, e.g., to control the size of the incision.

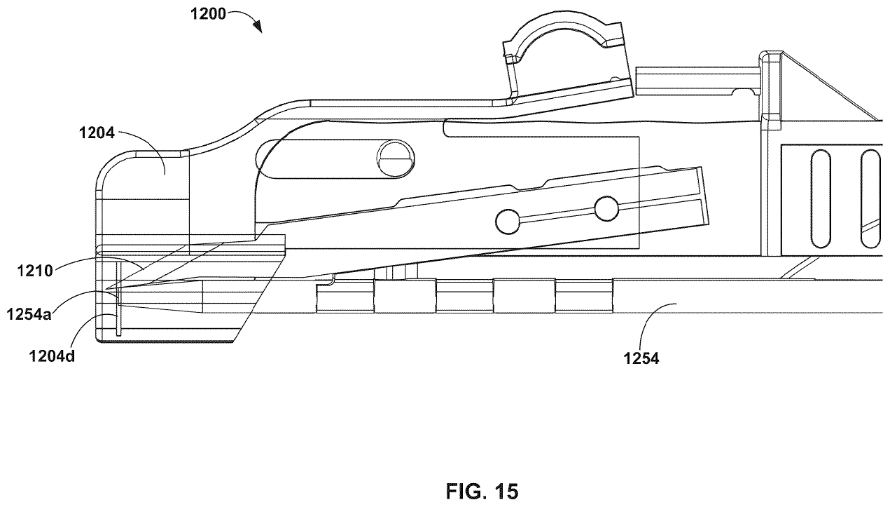

The guard 1204 includes a tab 1205 (e.g., actuator, etc.) that acts as a safety to prevent undesirable exposure of the cutting element 1210. The tab 1205 is a flexible bent portion that is biased away from the body 1202 and configured to engage a distal tip 1208a of a stop 1208 so that the guard 1204 is prevented from moved from the extended position as seen in FIG. 13A. In some embodiments, the tab 1205 is integrally formed with the guard 1204. To operate the guard 1204 between the extended position and the retracted position, the tab 1205 can be pressed toward the body 1202 (e.g., depressed) so that the tab 1205 can be pulled proximally under the stop 1208 as seen in FIG. 13B. The guard 1204, via the tab 1205, is then translated proximally along the slot 1204a to the extended position as seen in FIG. 13C. In the extended position, a protrusion 1205a (e.g., detent, etc.) on or near the proximal end of the tab 1205 engages a slot 1208b (e.g., receptacle, etc.) configured to accept the protrusion 1205a. The bias of the tab 1205 pushes the protrusion 1205a into the slot 1208b thus locking the guard 1204 in the extended position. The cutting device 1200 can then be used to form an incision and after the incision has been made, the guard 1204 can be returned to the extended position by again pressing down on the tab 1205 and pushing the tab 1205 distally away from the stop 1208 as in FIG. 13A. As seen in FIG. 15, which depicts a close up side view of the cutting device 1200, the guard 1204 (shown as transparent) can include an indicator line 1204d that is configured to indicate the distal end 1254a of the shaft 1254 when the shaft 1254 is coupled to the cutting device 1200. The indicator line 1204d is a visual marking that can be used by a user to align the cutting device 1200.

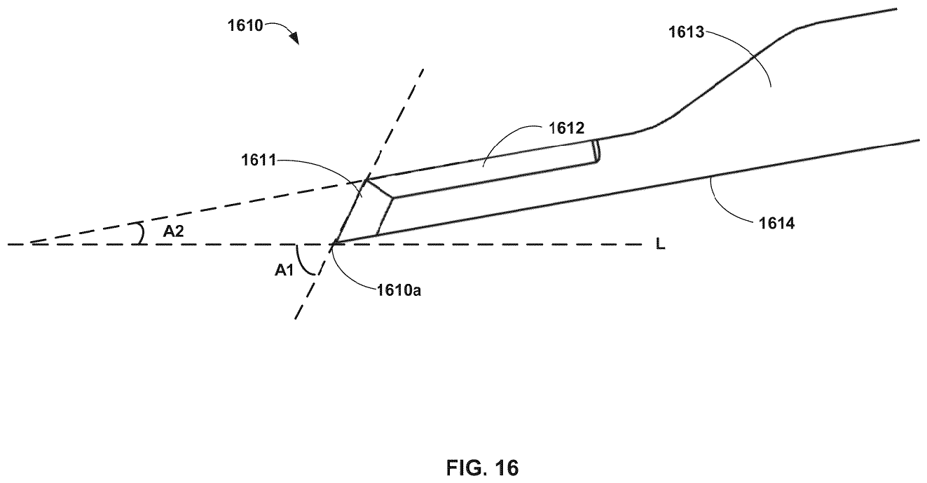

FIG. 16 depicts a cutting element 1610 (e.g., functionally and/or structurally similar to any of the cutting elements described herein such as the cutting element 110 of FIG. 1) with dual cutting edges 1611, 1612, according to embodiments. The first cutting edge 1611 is at the distal end of the cutting element 1610 and forms an angle A1 with a longitudinal axis L. The longitudinal axis is an axis defined by the body of any of the cutting elements described herein. In some embodiments, the angle A1 is between about 45 degrees and about 5 degrees, inclusive of all ranges and values therebetween. In some embodiments, the angle A1 is less than about 35 degrees. In some embodiments, the angle A1 is greater than about 5 degrees. In some embodiments, a smaller angle A1 is desirable as the surface area between the skin and the blade is smaller, thus inducing greater force and allowing to cut through skin with greater ease.

The second cutting edge 1612 extends proximally away from the first cutting edge. The second cutting edge 1612 defines an angle A2 between the second cutting edge 1612 and the longitudinal axis L. In some embodiments, the angle A2 is different than the angle A1. In some embodiments, the angle A2 is less than the angle A1. The configuration of the first cutting edge 1611 and the second cutting edge 1612 allows for the cutting element 1610 to cut deeper into the epidermis/dermis. In some embodiments, the incisions is between about 3 mm to 5 mm below the epidermis. In some embodiments, the cutting element 1610 is configured to form incisions up to and less than about 20 mm deep. In some embodiments, the cutting element 1610 is configured to form incisions of less than about 15 mm. The cutting element 1610 further includes a third edge 1614, which, in some embodiments, is not a cutting edge. The third edge 1614 is opposite the first cutting edge 1611 and the second cutting edge 1612. In some embodiments, the second cutting edge 1612 and the third edge 1614 extend substantially parallel to one another.

The body 1613 extends proximally away from the second cutting edge 1612. The body 1613 may be thicker than the portion including the first cutting edge 1611 and the second cutting edge 1612 as to provide the cutting element with support and structure. In some embodiments, the body 1613 can includes features (e.g., opening, slots, etc.) that allow for the body 1613 to couple to a cutting device, such as the cutting devices described herein.

While various embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto; and that embodiments may be practiced otherwise than as specifically described and claimed without departing from the scope and spirit of the present disclosure. Embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope and spirit of the present disclosure.

As used herein, the terms “about” and/or “approximately” when used in conjunction with values and/or ranges generally refer to those values and/or ranges near to a recited value and/or range. In some instances, the terms “about” and “approximately” may mean within ±10% of the recited value. For example, in some instances, “approximately a diameter of an instrument” may mean within ±10% of the length of the instrument. The terms “about” and “approximately”may be used interchangeably.

Also, various concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

1-20. (canceled)

21. An apparatus, comprising:
a cutting device including:
a body defining a longitudinal axis;
a cutting element coupled to a distal end of the body and extending distally therefrom, the cutting element including a cutting edge that is angled with respect to the longitudinal axis by an angle of less than about 45 degrees;
a plurality of couplers configured to reversibly couple the cutting device to a dilator at a predetermined position relative to the dilator, the cutting device and the dilator when coupled being advanceable along a guidewire that has been inserted into a puncture site formed in tissue,
the plurality of couplers, when the cutting device and the dilator are being advanced along the guidewire, being configured to maintain the cutting device at the predetermined position in which a distal end of the cutting element is positioned distal to a distal end of the dilator and angled toward the guidewire, such that the cutting element is configured to contact tissue followed by the dilator to form an incision in the tissue that is adjacent to the puncture site and the guidewire; and
a guard slidably coupled to the body, the guard configured to move between an extended position in which the guard covers the cutting element and a retracted position in which the cutting edge of the cutting element is exposed and configured to form the incision in the tissue.

22. The apparatus of claim 21, wherein the cutting device further includes an actuator coupled to the guard, the actuator configured to be depressed to allow the guard to move from the extended position to the retracted position.

23. The apparatus of claim 22, wherein the actuator, after the guard has been moved from the extended position to the retracted position, is configured to be released such that a detent disposed on the guard is configured to lock into a receptacle formed in the body.

24. The apparatus of claim 21, wherein the angle of the cutting edge is less than about 35 degrees.

25. The apparatus of claim 21, wherein the angle of the cutting edge is greater than about 5 degrees.

26. The apparatus of claim 21, wherein the cutting edge is a first cutting edge, and the cutting element includes a second cutting edge, the second cutting edge extending proximally from the first cutting edge at an angle relative to the longitudinal axis that is different than the angle of the first edge.

27. The apparatus of claim 26, wherein the angle of the second cutting edge is less than the angle of the first cutting edge.

28. The apparatus of claim 21, wherein the cutting element is configured to form the incision having a length of less than about 12.5 mm.

29. The apparatus of claim 21, wherein the body includes a distal surface that is configured to contact the tissue and to prevent further insertion of the cutting element into the tissue, thereby controlling a depth of insertion of the cutting element.

30. The apparatus of claim 29, wherein the depth of insertion of the cutting element is less than about 20 mm.

31. The apparatus of claim 21, wherein the plurality of couplers includes at least three couplers.

32. The apparatus of claim 31, wherein each coupler from the plurality of couplers includes a leg that is configured to extend around one side of the dilator, with legs of adjacent couplers from the plurality of couplers being offset from one another.

33. The apparatus of claim 32, wherein the legs of the plurality of couplers are resiliently deformable.

34. The apparatus of claim 21, wherein the cutting edge is an outer edge that faces away from the dilator when the cutting device is coupled to the dilator via the plurality of couplers, the apparatus further comprising:
the dilator, the distal end of the dilator having a tapered shape,
the cutting element further including an inner edge that is configured to substantially follow the tapered shape of the distal end of the dilator when the cutting device is coupled to the dilator.

35. An apparatus, comprising:
a cutting device including:
a body defining a longitudinal axis;
a plurality of couplers configured to reversibly couple the cutting device to a dilator at a predetermined position relative to the dilator, the cutting device and the dilator when coupled being advanceable along a guidewire that has been inserted into a puncture site formed in tissue;
a cutting element coupled to a distal end of the body and extending distally therefrom, the cutting element including first and second edges that are angled with respect to the longitudinal axis, the first edge being a cutting edge that faces away from the dilator when the cutting device is coupled to the dilator, the second edge being configured to substantially follow a tapered shape of a distal end of the dilator, the first and second edges terminating at a sharp distal end,
the plurality of couplers, when the cutting device and the dilator are being advanced along the guidewire, being configured to maintain the cutting device at the predetermined position in which the distal end of the cutting element is positioned distal to the distal end of the dilator with the cutting edge and the inner edge being angled toward the guidewire, such that the cutting element is configured to contact tissue followed by the dilator to form an incision in the tissue that that is adjacent to the puncture site and the guidewire; and
a guard slidably coupled to the body, the guard configured to move between an extended position in which the guard covers the cutting element and a retracted position in which the first edge is exposed and configured to form the incision in the tissue.

36. The apparatus of claim 35, wherein the first edge is angled with respect to the longitudinal axis by a first angle, and the second edge is angled with respect to the longitudinal axis by a second angle that is less than the first angle.

37. The apparatus of claim 36, wherein the cutting element further includes a third edge, the third edge being angled with respect to the longitudinal axis at a third angle that is substantially equal to the second angle such that the second and third edges extend parallel to one another.

38. The apparatus of claim 35, wherein the first edge is angled with respect to the longitudinal axis by an angle that is between about 5 degrees and about 45 degrees.

39. The apparatus of claim 35, wherein the cutting element is configured to form the incision having a length of less than about 12.5 mm.

40. The apparatus of claim 35, wherein the body includes a distal surface that is configured to contact the tissue and to prevent further insertion of the cutting element into the tissue, thereby controlling a depth of insertion of the cutting element.

41. The apparatus of claim 40, wherein the depth of insertion of the cutting element is less than about 20 mm.

42. The apparatus of claim 35, wherein the cutting device further includes an actuator coupled to the guard, the actuator configured to be depressed to allow the guard to move from the extended position to the retracted position.

43. The apparatus of claim 35, wherein the plurality of couplers includes at least three couplers, and each coupler from the plurality of couplers includes a leg that is configured to extend around one side of the dilator, with legs of adjacent couplers from the plurality of couplers being offset from one another.

44. The apparatus of claim 35, wherein the distal end of the cutting element, when the cutting device is coupled to the dilator at the predetermined position, extends past the distal end of the dilator by a distance of between about 0.5 mm and about 5 mm.

45. The apparatus of claim 35, wherein the guard includes an indicator that is configured to be aligned with the distal end of the dilator when the cutting device is coupled to the dilator to set the predetermined position.

46. The apparatus of claim 45, wherein at least a portion of the guard is formed of a translucent or transparent material such that the distal end of the dilator is visible through the portion of the guard to facilitate alignment of the distal end of the dilator with the indicator.

47. A method, comprising:
advancing a dilator and a cutting device coupled thereto, over a guidewire positioned through a puncture site formed in tissue, toward the puncture site, the cutting device including a cutting element configured to cut the tissue;
moving a guard of the cutting device from an extended position in which the guard is covering the cutting element to a retracted position in which a portion of the cutting element including a cutting edge is exposed, the cutting edge being at an angle relative to a longitudinal axis of the cutting device and extending distally beyond a distal end of the dilator; and
inserting, after moving the guard to the retracted position and by further advancing the cutting device and the dilator toward the tissue, a distal end of the cutting edge followed by the distal end of the dilator into the tissue to form an incision adjacent to the puncture site and the guidewire and sized to receive a surgical instrument.

48. The method of claim 47, wherein the cutting device includes a body including a distal surface configured to limit a depth of insertion of the cutting element into the tissue, and the distal end of the cutting element and the dilator are inserted into the tissue until the distal surface contacts a region of tissue near the puncture site and prevents further insertion of the cutting element into the tissue.
